(19) United States
(12) Patent Application Publication
(10) Pub. No.: US 2024/0343679 A1
(43) Pub. Date: Oct. 17, 2024

(54) Artificial oxygen carrier blood product

(71) Applicant: Andre Danesh, Brookline, MA (US)

(72) Inventor: Andre Danesh, Brookline, MA (US)

(21) Appl. No.: 18/445,115

(22) Filed: Apr. 17, 2023

(51) Int. Cl. C07C 229/76; A61P 7/00

(52) U.S. Cl. CPC C07C 229/76; A61P 7/00

(57) ABSTRACT

The invention comprises a process and composition relating to the use of an artificial blood that is a pharmaceutically acceptable solution of an iron amino acid chelate. In a preferred embodiment of the invention, the amino acid chelate is formed from a glycinate from pyridine or a bis pyridine. In use, the amino acid chelate is dissolved in a suitable carrier fluid such as an isotonic saline solution and transfused into an individual suffering trauma from loss of blood.

### FIELD OF THE INVENTION

The invention relates generally to the development of artificial blood compositions. More specifically, the invention relates to pharmaceutically acceptable amino acid chelates for use in the formulation of an artificial blood product.

### BACKGROUND

Circumstances such as accidents, surgery, traumatic hemorrhagic shock, and other casualties cause major blood loss. Allogenic blood transfusion can be resuscitative for such conditions; however, it has numerous ambivalent effects, including supply shortage, needs for more time, cost for blood grouping, the possibility of spreading an infection, and short shelf-life. Hypoxia or ischemia causes heart failure, neurological problems, and organ damage in many patients. To address this emergent medical need for resuscitation and to treat hypoxic conditions as well as to enhance oxygen transportation, researchers aspire to achieve a robust technology aimed to develop safe and feasible blood substitutes, or artificial blood, for effective oxygen transport.

Efforts to produce artificial blood were spurred on by the military in World Wars I and II and, more recently, by the discovery in the early 1980s that HIV could be transmitted by blood transfusion. Blood is now safe, thanks to improved collection and screening by blood banks. But it still has to be cross-matched and can be stored for only a few weeks before it has to be discarded. If a solution that could replace blood were immediately available, if it were completely safe, and if it could be stored for long periods; it would be extremely useful in emergencies, disaster and wars—not to mention in countries where blood is not collected and stored as it is in the U.S and Europe.

Artificial blood is designed to transport oxygen and carbon dioxide through the body. No substitutes have yet been invented that can replace the other vital functions of blood: coagulation and immune defense. Therefore, the replacement solutions being developed today are more accurately described as oxygen carriers. There are basically two types of oxygen carriers, which differ in the way they transport oxygen. One is based on perfluoro chemicals, the other on hemoglobin.

The following discussion relates to hemoglobin blood substitutes. Hemoglobin-based oxygen carriers (HBOCs) utilize the same oxygen-carrying protein molecule found in blood where the oxygen bonds chemically to hemoglobin. HBOCs differ from red blood cells in that the hemoglobin is not contained within a membrane. The membrane of a red blood cell contains the antigen molecules that determine the ‘type’ of the blood (A, B, AB or O). Because HBOCs have no membranes, they do not need to be cross-matched by type and can be given to any patient without previous testing. In addition, these artificial oxygen carriers can be stored for long periods of time greatly simplifying the work of the blood bank. Best of all, HBOCs can be used in situations and locations where real blood is not available, as at disaster sites, underdeveloped countries or battle zones. Moreover, unlike red blood cells, blood substitutes can be pasteurized, filtered and chemical-cleansed to make them sterile. These procedures remove microorganisms responsible for diseases such as AIDS and hepatitis. Because the substitutes do not have cell membranes with blood-group antigens, cross-matching and typing are not required before use. This saves time and facilities and allows on-the-spot transfusion. Furthermore, blood substitutes can be stored for more than one year, as compared with about one month for donor blood stored using standard methods.

Two problems arise when hemoglobin is removed from the red blood cells and these problems account for the large amount of scientific research that has been conducted so far in this area. First, the red cell membrane protects hemoglobin from degradation and protects tissues from the toxic effects of free hemoglobin. Second, when oxygen is delivered by a cell-free carrier instead of red blood cells, complex biological mechanisms alter the flow through the smallest blood vessels (the arterioles and capillaries). Major advances have been made in overcoming both of these problems, and several HBOC products are now in advanced human trials. It is anticipated that within the next several years, the first of these products will become available to physicians for use in patients.

Hemoglobin is the protein in red blood cells that is responsible for carrying oxygen from the lung to the other tissues. Therefore, current methods to formulate blood substitutes are to use hemoglobin extracted from red blood cells. However, raw hemoglobin extracted from red blood cells cannot be used as a blood substitute. Each hemoglobin molecule consists of four subunits, known as tetramers. When infused into the body, a hemoglobin molecule breaks down into potentially toxic half molecules, or dimers. There are also other problems related to hemoglobin in free solution. The challenge those engaged in research based upon hemoglobin is to modify the hemoglobin to allow it for use as a blood substitute.

First generation hemoglobin blood substitutes rely on molecular modifications of hemoglobin, either by chemically cross-linking the molecules or by modifying them using recombinant DNA technology. So-called bifunctional agents can cross-link the hemoglobin molecules to one another to form polyhemoglobin. The basic idea of cross-linked hemoglobin and encapsulated hemoglobin dates back to the 1960s (T. M. S. Chang in Science, Vol. 146, page 524; 1964, and H. F. Bunn and J. H. Jandl in the Transactions of the Association of American Physicians, Vol. 81, page 147; 1968). The cross-linked hemoglobin molecules are stable and do not break down. Some bifunctional agents can also cross-link each hemoglobin molecule internally to prevent its breakdown into dimers. Recombinant technology applied to the bacterium E. coli can produce altered hemoglobin molecules that do not break down into half molecules. Hemoglobin can also be cross-linked to soluble polymers to form so-called conjugated hemoglobin. All the above modifications also result in blood substitutes that have a greater ability to release oxygen to the tissues than do red blood cells. Clinical trials in humans are ongoing using products from a number of companies. In the case of polyhemoglobin, Northfield is now in Phase III (large-scale efficacy) clinical trials that infuse up to 5,000 milliliters of blood substitutes into surgical patients. The company is using pyridoxalated glutaraldehyde cross-linked human hemoglobin. Biopure is in Phase II (small-scale efficacy) clinical trials using pyridoxalated glutaraldehyde cross-linked bovine hemoglobin. Hemosol is in Phase II clinical trials in surgical patients, using a new cross-linker to form a molecule known as o-raffinose cross-linked human polyhemoglobin. In the case of intramolecularly cross-linked hemoglobin, Baxter is now in Phase III clinical trials in a large number of surgical patients; the company is using Diaspirin cross-linked human hemoglobin. Somatogen is now deep into their Phase II clinical trials with their recombinant human hemoglobin. In conjugated hemoglobin, Enzon is now in Phase II clinical trials, and Apex is now in Phase I (safety) clinical trials.

Further discussion of artificial blood may be found in the following publications: Hemoglobin-Based Red Cell Substitutes. Robert M. Winslow. Johns Hopkins University Press, 1992; Blood Substitutes—A Moving Target. Robert M. Winslow in Nature Medicine, Vol. 1, No. 11, pages 1212-1215; 1995; and Blood Substitutes. Robert M. Winslow in Science & Medicine, Vol. 4, No. 2, pages 54-63; 1996; Artificial Oxygen Carriers and Red Blood Cell Substitutes: An Historic Overview and Recent Developments Toward Military and Clinical Relevance: Christopher Bialas, Christopher Moser, C. Sims, Medicine The Journal of Trauma and Acute Care Surgery, 2019; From hemoglobin allostery to hemoglobin-based oxygen carriers, S. Faggiano, L. Ronda, A. Mozzarelli, Biology, Medicine, Molecular aspects of medicine, 2021.

Though efforts to develop an artificial blood based upon hemoglobin are in clinical trials, the required hemoglobin needed for the formulation of an artificial blood product relies upon using either native or recombinant human hemoglobin, modified forms of human hemoglobin or modified forms of hemoglobin from other species. Unmodified hemoglobin can be used as an oxygen therapeutic; however, it can bind NOx and causes severe vasoconstriction and hypertension. As a consequence of its molecular weight, hemoglobin can cause significant toxicities, especially to the kidney where it clogs the glomerular apparatus. As a consequence, most hemoglobins tested in humans are modified to prolong their half-life and reduce their toxicity. Consequently, hemoglobin as an artificial blood is costly and difficult to acquire and store.

### SUMMARY OF THE INVENTION

The subject invention involves the use of non-hemoglobin iron amino acid chelates as a component of an artificial oxygen carrier replacement for blood. In a preferred embodiment of the invention, the amino acid chelate is a glycinate of an amino acid or a chelate of a pyridine. In use, the amino acid chelate is dissolved in a suitable liquid carrier such as an isotonic saline solution and is transfused into an individual suffering trauma due to loss of blood.

### DESCRIPTION OF THE PREFERRED EMBODIMENTS

Amino acid chelates are well known in the art and are of significant importance for use in many applications, fields, and industries such as food, animal feed, supplement, pharmaceutical production, and as bio-fertilizers. For example, fertilizers of inorganic mineral structure are hardly diffused from the leaf surface into the plant, while chelated minerals with amino acids provide a great advantage in increasing the absorption efficiency and translocation of minerals within the plant. Nanotechnology increases the application efficiency of metal-amino acid complexes. Using nano fertilizers in plants is of critical importance due to their unique properties in size and increased surface areas. It releases the nutrients on demand and regulates plant growth (such as wheat, rice, barley, and rapeseed plants).

Metal amino acid chelating complexes have found extensive applications in various fields of human interest. Amino acids are the building blocks of proteins, which are vital for many bodily functions. When a person eats a food that contains a protein, their digestive system breaks the protein down into amino acids. There are 20 amino acids, but nine are essential, meaning it is necessary to acquire them from food or supplements. The body combines the amino acids in various ways to carry out bodily functions. A healthy body can manufacture needed amino acids, so these do not usually need to enter the body through the diet. These amino acids build muscles, enable chemical reactions in the body, transport nutrients, prevent illness, and carry out other functions. Amino acid deficiency can result in decreased immunity, digestive problems, depression, fertility issues, lower mental alertness, slowed growth in children, and many other health issues. To treat amino acid deficiency, amino acids are used as supplements and these supplements are often administered in the form of an amino acid chelate.

Amino acid chelates are also used in additional applications such as water softeners. In addition, they are included as ingredients in many commercial products such as shampoos and food preservatives and control heavy metal pollution in aquacultures.

As a consequence of the extensive use of amino acid chelates, methods for the synthesis of such chelates are well known in the art and are not considered to be a part of the invention disclosed herein. The structure, chemistry and the bioavailability of amino acid chelates are described in a variety of documents, for example, Ashmead et al., Chelated Mineral Nutrition, (1982), Chas. C. Thomas Publishers, Springfield, III.; Ashmead et al., Intestinal Absorption of Metal Ions, (1985); Ashmead et al., Foliar Feeding of Plants with Amino Acid Chelates, (1986); Ashmead et al, U.S. Pat. No. 4,020,158 granted Aug. 26, 1977; U.S. Pat. No. 4,167,564; Jensen, U.S. Pat. No. 4,216,143 granted Sep. 11, 1979; Mayo, U.S. Pat. No. 4,721,644 granted Jan. 26, 1988; Ashmead, U.S. Pat. No. 4,599,152 granted Jul. 8, 1986; Ashmead, U.S. Pat. No. 4,774,089 granted Sep. 27, 1988; Ashmead, U.S. Pat. No. 4,830,716 granted May 6, 1989; Ashmead et al, U.S. Pat. No. 4,863,898 granted May 9, 1989; and Ashmead et al, U.S. Pat. No. 4,725,427 granted Feb. 16, 1988, each incorporated herein by reference for their teachings regarding the synthesis of these chelates and their uses.

As is known in the art, amino acid chelates are generally made by reacting alpha-amino acids and metal ions, where the metal ion has a valence of 2 or more, to form a ring structure in the form of a chelate. In such a reaction, the cationic charge of the metal ion is neutralized by the free amino group or carboxyl group of the alpha-amino acid. One such representative metallic amino acid chelate and representative reaction to form this chelate is represented by the formula below:

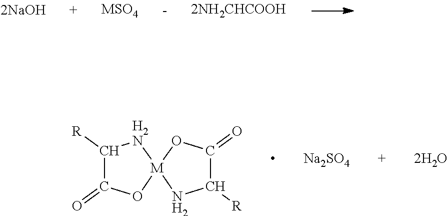

wherein M is a divalent metal ion, and R is a side chain of naturally occurring amino acids or peptides. The metal ion M includes, for example, iron, zinc, manganese, magnesium, copper, and calcium. Iron chelates are the chelate most often used for treatment of humans. Other amino acids that can be converted to chelates include, for example, alanine, arginine, asparagine, aspartate, cysteine, glutamate, glutamine, glycine, proline, serine, and tyrosine.

Though the current invention contemplates the use of amino acid chelates for use in an artificial oxygen carrier blood composition, the preferred chelates are iron chelates of a glycinate or an iron chelate of a pyridine.

With respect to the iron chelates of a glycinate, the most preferred amino acid chelates in accordance with this invention are those prepared using the glycinates disclosed by Israily in U.S. Pat. No. 3,567,752 granted Mar. 2, 1971 and incorporated herein by reference. This patent discloses a method for forming amino acid chelates using a glycine conforming to one of the following structural formulae:

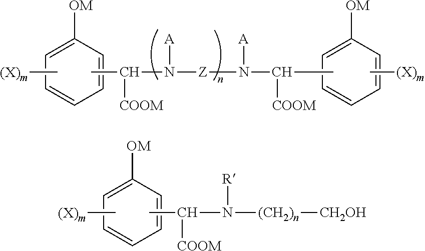

where; in the above formulas, where applicable; A is selected from the group of hydrogen and —RCH—CH2OH provided that at least one A is —RCH—CH2OH.; R is selected from the group of hydrogen and alkyl having up to 13 carbon atoms; R is selected from the group of R. and alkanol having up to 13 carbon atoms; Z is alkylene having from 2 to 4 carbon atoms; X is selected from the group of alkyl and alkoxy having up to 13 carbon atoms, hydroxy, chloro and bromo; M is selected from the group of hydrogen, sodium, potassium, ammonium and amine derivatives; m is a whole number equal to from 0 to the number of replaceable hydrogen atoms on the benzene nucleus and n is a whole integer equal to from 1 to 5.

Example 2 of U.S. Pat. No. 3,567,752 illustrates a procedure for forming a chelate used as a component of an artificial blood composition in accordance with the invention. This example is reproduced herein for its teachings of the formation of the preferred chelates used to form an artificial blood component.

### Example II.—Preparation of the iron chelates of N,N′-ethylene bis(2-(o-hydroxyphenyl)-2 hydroxyethylglycine

(A) Ferric chelate process.—A Solution comprising 44.4 grams (0.10 mole) of the free acid (E.H.P.H.E.G.) of Example I in 200 ml. water was prepared. To this solution was added 40.4 grams (0.10 mole) of ferric nitrate. The solution turned purple. Thereafter, 12 grams (0.30 mole) of NaOH were added and the monohydrogen ferric (E.H.P.H.E.G.) chelate separated out. The chelate was filtered from the solution and dried in a vacuum oven at 50 C. Thereafter, 26.05 grams of the chelate (0.05 mole) were mixed with 100 ml. of water followed by the addition of 2 grams of NaOH to form a deep red solution. This was evaporated and the residue was dissolved in methanol. The mixture was filtered, methanol removed and the monosodium ferric (E.H.-P.H.E.G.) recovered. This chelate is very soluble in water and methanol.
Analysis.-Calculated for Ca2H2NONaFe (percent): N, 5.35. Found: (percent) N, 5.39.
Potassium hydroxide or ammonium hydroxide may be substituted for sodium hydroxide to form the corresponding mono-potassium or mono-ammonium ferric (E.H.P.H.E.G.) chelate.

Following the procedures of Example 2 of said patent and using ferric chloride and sodium hydroxide as reactants, a ferric chelate of N,N′-ethylene bis-(2-(o-hydroxyphenyl)-2-hydroxyethylglycine can be formed having the structure:

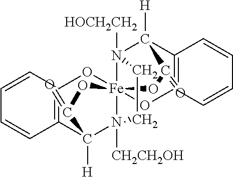

Additional amino acid chelates may be formed from the precursors following the procedures disclosed in said patent. Other metals salts, inclusive of salts of calcium, zinc, magnesium, copper, iron, cobalt, manganese, chromium, may be substituted for the ferric chloride to obtain the corresponding metal chelate of ferric chelate of N,N′-ethylene bis-(2-(o-hydroxyphenyl)-2-hydroxyethylglycine.

Iron chelates formed from pyridine. dipyridine and their derivatives are also preferred in accordance with this invention. The pyridine and dipyridine starting materials may be represented by the following formulae:

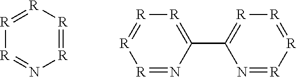

where at least one R is an alkyl amine. The alkyl amine may then be reacted with a sodium salt of methyl chloride which is then reacted with hydrochloric acid to form the pyridine carboxylic acid.

As discussed above, the chelate is formed from a carboxyl acid substituted pyridine or a carboxylic acid bis pyridine—for example:

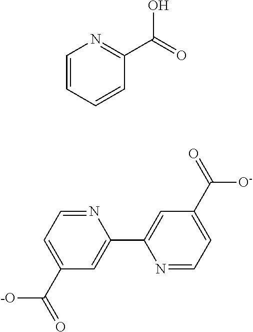

Methods for formation of metal chelates of carboxylic acid substituted pyridine and bis pyridines are known in the art and disclosed in numerous publications inclusive of Methyl-Hydroxypyridinecarboxylic Acids as Possible Bidentate Chelating Agents for Aluminium(III): Synthesis and Metal-Ligand Solution Chemistry, VB Di Marco et al, European Journal of Inorganic Chemistry, VB Di Marco et al, 2002—Wiley Online Library, Volume2002, Issue10. Pages 1284-1293; Evaluation of 1-methyl-3, 4-hydroxypyridinecarboxylic acids as possible bidentate chelating agents for iron (III): Metal-ligand solution chemistry, VB Di Marco et al, Polyhedron 26(13):3227-3232, DOI:10.1016/j.poly.2007.02.026, August 2007; Different approaches to the study of chelating agents for iron and aluminium overload pathologies, Guido Crispon et al, Anal Bioanal Chem. 2013 January; 405(2-3):585-601, doi: 10.1007/s00216-012-6468-7; 1, 6-Dimethyl-4-hydroxy-3-pyridinecarboxylic acid and 4-hydroxy-2-methyl-3-pyridinecarboxylic acid as new possible chelating agents for iron and aluminium, Dean et al, Dalton Trans. 2009 Mar. 14; (10):1815-24. doi: 10.1039/b819148d. Epub 2009 Jan. 27. VB Di Marco et al, Inorganica, Chimica Acta, Volume 357, Issue 12, 10 Sep. 2004, Pages 3753-3758, and Neutral-Ligand Complexes of Bis(imino)pyridine Iron; and Synthesis, Structure, and Spectroscopy, SC Bart et al, norg. Chem. 2007, 46, 17, 7055-7063, Publication Date: Jul. 26, 2007, https://doi.org/10.1021/ic700869h, each incorporated herein by reference for the teachings of amino acid complexes formed from pyridine based carboxylic acids.

Additional references relevant to the formation of such chelates include Neutral-ligand complexes of bis (imino) pyridine iron: Synthesis, structure, and spectroscopy, SC Bart, E Lobkovsky, E Bill, K Wieghardt, Chemistry, Inorg. Chem. 2007, 46, 17, 7055-7063, Publication Date: Jul. 26, 2007; Hydroxypyridinones with enhanced iron chelating properties. Synthesis, characterization and in vivo tests of 5-hydroxy-2-(hydroxymethyl)pyridine-4(1H)-one, J. I. Lachowicz, V. M. Nurchi, G. Crisponi, M. G. Jaraquemada-Pelaez, M. Arca, A. Pintus, M. A. Santos b, C. Quintanova, L. Gano, Z. Szewczuk M. A. Zoroddu, M. Peana, A. Dominguez-Martin and D. Choquesillo-Lazarte, Dalton Trans., 2016, 45, 6517-6528; and Asymmetric diimine pyridine iron or cobalt complex catalyst; and Preparation Method and Application Thereof, CN102464677B granted Aug. 13, 2014, each incorporated herein for their teachings of pyridine and bis pyridine metal, inclusive of iron, chelates.

A preferred amino acid chelate formed from a pyridine is disclosed in Souchay P; Israily N, Gouzerh P. No 667. —Preparation et etude en solution de nouveaux amino diacides complexants [No. 667. —Preparation and study of new amino diacid complexes in solution]. Bull Soc Chim Fr. 1966 December; 12:3917-26. French. PMID: 5992497. 1966 December; 12:3917-26, incorporated herein by reference where the metal used to form the chelate is Fe. The chelates formed in accordance with this publication would have formulas such as:

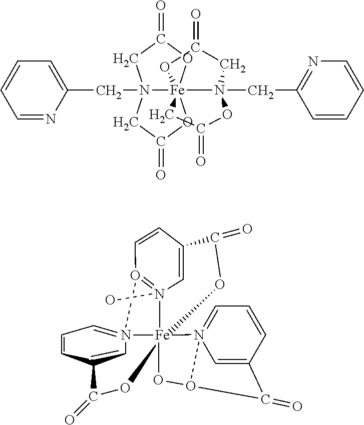

where said formulas are not intended to specify a particular stereochemistry.

Additional useful amino acid chelates in accordance with this invention include iron chelates formed from amino acids that are chains typically attached to hemoglobin, for example amino acid chelates of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

In various embodiments of the invention, the composition includes a pharmaceutically acceptable carrier, such as a diluent fraction comprising a salt. The salt can be selected from essentially any salt, though the salts presently preferred are salts that are pharmaceutically acceptable for delivery to mammals. In various embodiments, the composition includes sodium chloride. The compositions of the invention are isotonic, hypertonic or hypotonic. In various embodiments, the composition is hypertonic. In an exemplary embodiment, the composition included sufficient sodium chloride to render it hypertonic. In other embodiments, the diluent is tonic phosphate buffered saline.

For use of a chelated amino acid in an oxygen carrier blood replacement formulation, pharmaceutical grade amino acid chelates, free of interfering anions are needed. A method for preparing a pharmaceutically acceptable amino acid chelate is disclosed by Ashmead, U.S. Pat. No. 4,830,716A, granted May 16, 1989 and incorporated herein by reference. In accordance with the process of this patent, active metals such as calcium, magnesium and manganese, when placed in the presence of amino acids, reduce the acidic amino acid proton to hydrogen which is given off as hydrogen gas. Iron, zinc and copper are not sufficiently reactive to cause this reduction to occur without the application of a slight potential to the metal. This may be done by the addition of electrolytes to an aqueous solution or by the application of an electric potential. These reactions all occur in an aqueous medium wherein the amino acid ligand has been at least partially dissolved. The most obvious evidence for the reaction of metals with amino acids, using glycine for purposes of illustration, is the disappearance of the metal and the ebullition of hydrogen according to the reactions:

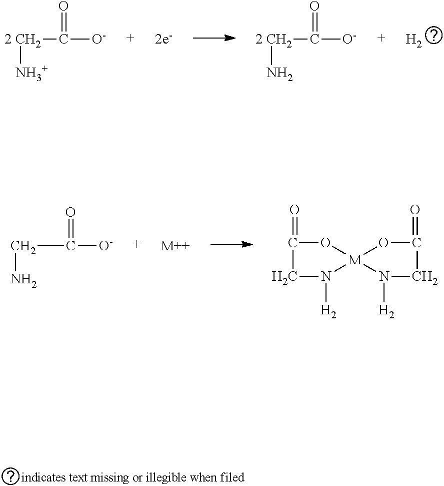

The pH of a glycine solution before reaction is about 6.0. When using calcium as the metal, the pH of the solution after the reaction, increases to about 9.4. With magnesium, the pH increases to about 10.5 and with manganese to about 9.0. A smaller pH increase, to about 7.8 is noted with iron. With zinc the pH increases to about 8.0 and with copper to about 7.5. When using copper, it is necessary to apply a potential electrically. Other metals may also be ionized, or oxidized, using the metal as an anode in an electrolytic cell. The cathode can be any inert material such as graphite. The reaction proceeds according to equations above. In the completed reaction, the metal is oxidized as the protons on the amino acids are reduced. Whether using an electrolytic method or not, a weak electrolyte such as citric or ascorbic acid may be added to the aqueous solution to promote the reaction.

Additional methods for forming pharmaceutically acceptable amino acid chelates suitable for use in accordance with the subject invention include: Asmead, CA1299812C granted 4/29/92; Ashmead, EP0256645A2 published Dec. 11, 1991; CN100540031C published Sep. 16, 2009; and Park, US20100093850A1 published Apr. 15, 2010.

The amino acid chelates of the present invention may be administered into the circulatory system by injecting the composition directly and/or indirectly into the circulatory system of the subject, by one or more injection methods. The preferred method is by intravascular injections, such as intravenous and intra-arterial injections. Accordingly, to prepare an artificial oxygen carrier blood composition using the amino acid chelates, the chelate would be dissolved in a pharmaceutically acceptable carrier. An intravenous solution would be an acceptable carrier. Of the various intravenous solutions, isotonic saline (0.9%) is recommended for use with the chelate. Other commonly used intravenous solutions might cause varying degrees of difficulty when mixed with the chelates. The concentration of the chelate in the saline solution from about 5 percent by weight up to 30 percent by weight but preferably varies between 10 percent by weight up to 25 percent by weight.

1. A process for treating an individual suffering trauma from loss of blood, said method comprising the step of infusing said individual with a pharmaceutically acceptable saline solution of a non-hemoglobin oxygen carrying iron amino acid chelate.

2. The process of claim 1 where the amino acid chelate is formed from a member selected from the group of: 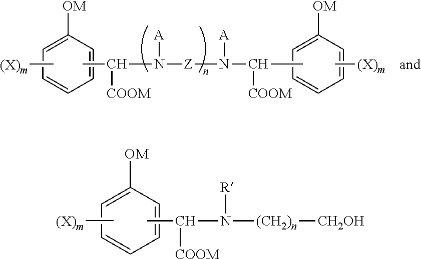
where; in the above formulas, A is selected from the group of hydrogen and —RCH—CH2OH provided that at least one A is —RCH—CH2OH.; R is selected from the group of hydrogen and alkyl having up to 13 carbon atoms; R′ is selected from the group of R, and alkanol having up to 13 carbon atoms; Z is an alkylene having from 2 to 4 carbon atoms; X is selected from the group of alkyl and alkoxy having up to 13 carbon atoms, hydroxy, chloro and bromo; M is selected from the group of hydrogen, sodium, potassium, ammonium and amine derivatives; m is a whole number equal to from 0 to the number of replaceable hydrogen atoms on the benzene nucleus and n is a whole integer equal to from 1 to 5.

3. The process of claim 2 where the amino acid chelate is formed from: 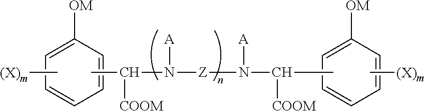
where A is selected from the group of hydrogen and —RCH—CH2OH provided that at least one A is —RCH—CH2OH, Z is an alkylene having from 2 to 4 carbon atoms; X is selected from the group of alkyl and alkoxy having up to 13 carbon atoms, hydroxy, chloro and bromo; M is selected from the group of hydrogen, sodium, potassium, ammonium and amine derivatives; m is a whole number equal to from 0 to the number of replaceable hydrogen atoms on the benzene nucleus.

4. The process of claim 2 where the amino acid chelate is formed from: 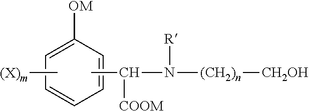
where R′ is selected from the group of alkanol having up to 13 carbon atoms; M is selected from the group of hydrogen, sodium, potassium, ammonium and amine derivatives; X is selected from the group of alkyl and alkoxy having up to 13 carbon atoms, hydroxy, chloro and bromo; and m is a whole number equal to from 0 to the number of replaceable hydrogen atoms on the benzene nucleus.

5. The process of claim 2 where the amino acid chelate is formed from:

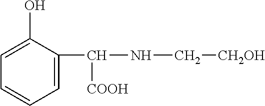 [SMILES: O=C(O)C(NCCO)c1ccccc1O]

6. The process of claim 2 where the amino acid chelate is formed from:

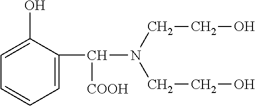 [SMILES: O=C(O)C(c1ccccc1O)N(CCO)CCO]

7. The process of claim 2 where the amino acid chelate is formed from:

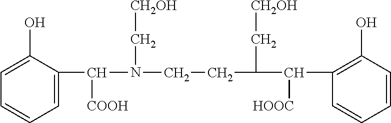 [SMILES: O=C(O)C(c1ccccc1O)C(CCO)CCN(CCO)C(C(=O)O)c1ccccc1O]

8. The process of claim 1 where the amino acid chelate is an iron formed from a member selected from the group of:

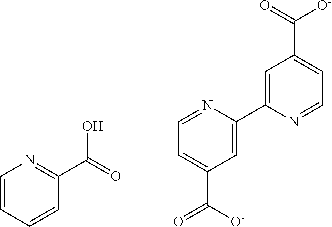 [SMILES: O=C(O)c1ccccn1 ; O=C([O-])c1ccnc(-c2cc(C(=O)[O-])ccn2)c1]

9. The process of claim 8 where the amino acid chelate is formed from

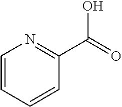

10. The process of claim 8 where the amino acid chelate is formed from

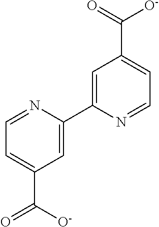 [SMILES: O=C([O-])c1ccnc(-c2cc(C(=O)[O-])ccn2)c1]

11. The process of claim 1 where the amino acid chelate is dissolved in said saline solution in a concentration of from 5 to 30% by weight.

12. The process of claim 11 where the amino acid chelate is dissolved in said saline solution in a concentration of from 10 to 25% by weight.

13. A non-hemoglobin oxygen carrying iron amino acid chelate blood composition comprising a saline solution of an iron amino acid chelate formed from a member selected from the group of: 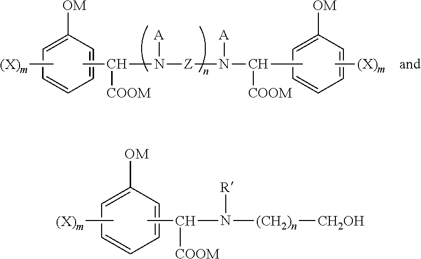
where; in the above formulas, A is selected from the group of hydrogen and —RCH—CH2OH provided that at least one A is —RCH—CH2OH.; R is selected from the group of hydrogen and alkyl having up to 13 carbon atoms; R is selected from the group of R, and alkanol having up to 13 carbon atoms; Z is an alkylene having from 2 to 4 carbon atoms; X is selected from the group of alkyl and alkoxy having up to 13 carbon atoms, hydroxy, chloro and bromo; M is selected from the group of hydrogen, sodium, potassium, ammonium and amine derivatives; m is a whole number equal to from 0 to the number of replaceable hydrogen atoms on the benzene nucleus and n is a whole integer equal to from 1 to 5.

14. The composition of claim 13 where the amino acid chelate is formed from: 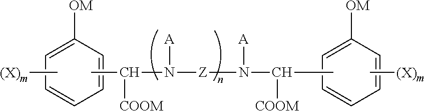
where A is selected from the group of hydrogen and —RCH—CH2OH provided that at least one A is —RCH—CH2OH, Z is an alkylene having from 2 to 4 carbon atoms; X is selected from the group of alkyl and alkoxy having up to 13 carbon atoms, hydroxy, chloro and bromo; M is selected from the group of hydrogen, sodium, potassium, ammonium and amine derivatives; m is a whole number equal to from 0 to the number of replaceable hydrogen atoms on the benzene nucleus.

15. The composition of claim 13 where the amino acid chelate is formed from: 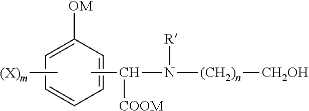
where R′ is selected from the group of alkanol having up to 13 carbon atoms; M is selected from the group of hydrogen, sodium, potassium, ammonium and amine derivatives; X is selected from the group of alkyl and alkoxy having up to 13 carbon atoms, hydroxy, chloro and bromo; and m is a whole number equal to from 0 to the number of replaceable hydrogen atoms on the benzene nucleus.

16. The composition of claim 13 where the amino acid chelate is formed from:

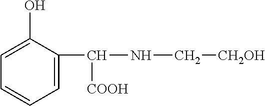 [SMILES: O=C(O)C(NCCO)c1ccccc1O]

17. The composition of claim 13 where the amino acid chelate is formed from:

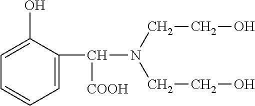 [SMILES: O=C(O)C(c1ccccc1O)N(CCO)CCO]

18. The composition of claim 13 where the amino acid chelate is dissolved in said saline solution in a concentration of from 5 to 30% by weight.

19. The composition of claim 18 where the amino acid chelate is dissolved in said saline solution in a concentration of from 10 to 25% by weight.

20. An oxygen carrying blood composition comprising a saline solution of an amino acid chelate where the amino acid chelate has the formula: 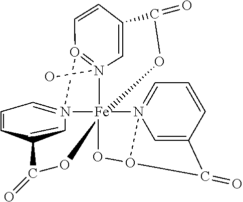
and the amino acid chelate is dissolved in said saline solution in a concentration of from 5 to 30% by weight.

21. An oxygen carrying blood composition comprising a saline solution of an amino acid chelate where the amino acid chelate has the formula: 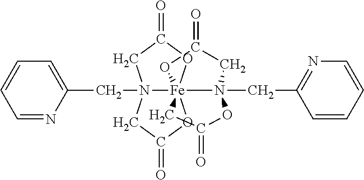
and the amino acid chelate is dissolved in said saline solution in a concentration of from 5 to 30% by weight.

22. A process for treating an individual suffering trauma from loss of blood, said method comprising the step of infusing said individual with a pharmaceutically acceptable saline solution of a non hemoblobin iron amino acid chelate.

23. The process of claim 22 where the chelate is present in solution in an amount of from 10 to 25% by weight.

24. An artificial blood composition comprising a pharmaceutically acceptable saline solution of a non hemoblobin iron amino acid chelate.

25. The artificial blood composition of claim 24 where the chelate is present in solution in an amount of from 10 to 25% by weight.
